(19) United States
(12) Patent Application Publication
(10) Pub. No.: US 2022/0148717 A1
(43) Pub. Date: May 12, 2022

(54) MEDICAL DEVICE REPORTING AND TRACKING

(71) Applicant: MDRisks, Inc., Boylston, MA (US)

(72) Inventors: Mohamed Yatim, Boylston, MA (US); Hussein Yatim, Waltham, MA (US); Hassan Yatim, Shrewsbury, MA (US); Alexander Duhani, Shrewsbury, MA (US); Riad Mortada, Foxborough, MA (US)

(21) Appl. No.: 17/521,643

(22) Filed: Nov. 8, 2021

Related U.S. Application Data
(60) Provisional application No. 63/111,288, filed on Nov. 9, 2020.

(51) Int. Cl. G16H 40/40; G16H 10/60; G06Q 50/04; G06K 7/14

(52) U.S. Cl. CPC G16H 40/40; G06K 7/1413; G06Q 50/04; G16H 10/60

(57) ABSTRACT

A medical device deployment tracking and reporting system for medical device usage and anomalies provides a coordinated and centralized approach to prompt identification and dissemination about adverse events resulting from deployed medical devices. Known conditions such as defects, recalls, and expirations can be made available prior to further usage of similar or related devices, and occurrences of new anomalies and possible adverse events can be reported to ensure timely dissemination to avert subsequent use of similar devices.

### RELATED APPLICATIONS

This patent application claims the benefit under 35 U.S.C. § 119(e) of U.S. Provisional Patent App. No. 63/111,288, filed Nov. 9, 2020, entitled “MEDICAL DEVICE REPORTING AND TRACKING” incorporated herein by reference in entirety.

### BACKGROUND

As medical and healthcare capabilities enlarge with expanding technology, available treatment and longevity options continue to become increasingly available. Emerging technology fuels development of more highly complex and effective medical devices for various diagnostic and/or corrective tasks. Since medical devices are typically extensions of or vehicles for treatment traditionally provided by a medical doctor, it is important to monitor, track and regulate these devices to avoid or mitigate adverse effects on the health of people, property, and environment. Reasonably foreseeable risks relating to medical devices are determined by the medical device manufacturer, of which the severity of the potential harms range from non-serious to serious injury/death. Although commercialization and business factors tend to naturally drive medical device manufacturers to improve and advance their technology, problems inevitably arise due to various factors. As with many manufactured commodities, anomalies such as defects and design errors occur, along with effects of misuse or improper operation attributable to human error.

### SUMMARY

A medical device deployment tracking and reporting system for medical device usage and anomalies provides a coordinated and centralized approach to prompt identification and dissemination about adverse events resulting from deployed medical devices. Known conditions such as defects, recalls, and expirations can be made available prior to further usage of similar or related devices, and occurrences of new anomalies and possible adverse events can be reported to ensure timely dissemination to avert subsequent use of similar devices.

Medical devices play a crucial role in healthcare—to diagnose and treat a wide range of patient conditions in order to improve the quality of life and at times, save them. These devices range from being as simple as a band-aid to a complex life-saving product such as a pacemaker. Complications or adverse events with medical devices are becoming more prevalent as the number and complexity of devices on the market has risen significantly and sales of medical devices have been growing at a global annual rate of about 3.87% from 2006 to 2016. The medical device industry experiences increasing pressures, including cost competitiveness, globalization, regulatory changes and constraints and supply chain tiering which can impact product quality and performance. The industry's vigorous growth and innovation places a new burden on quality systems. Evidence of this includes an increase in medical device related serious patient adverse events reported to the U.S. Food and Drug Administration (FDA). Market research indicates that reports of adverse events is rising faster than the overall medical device market.

Configurations herein are based, in part, on the observation that a multitude of deployed medical devices are invoked for various functions ranging from non-invasive diagnoses to potentially invasive and surgical uses. Unfortunately, conventional approaches to medical device deployment suffer from the shortcoming that occurrences of a defect or anomaly in a device may not be readily communicated to or made apparent to other users of similar devices. Manufacturing errors and design defects likely affect an entire product line, while aspects such as device recalls and expirations may not be readily apparent to a user of such devices. Accordingly, configurations herein substantially overcome the above shortcomings of conventional medical device deployment by providing a registration and tracking system that scans a unique, bar coded identifier affixed to each device package prior to usage, and references a central database to immediately warn the eminent user of a recall, defect, expiration or other anomaly which might affect safe usage or administration of the medical device.

Configurations herein allow for tracking defects and anomalies in deployed medical devices by accumulating a repository of anomalies indicative of deployed medical appliances and receiving an identifier indicative of a deployed medical appliance. The received identifier is indicative of the device vendor, a model number, and one or more of the following production identifiers: the lot number within which the device was manufactured, serial number of the specific device, expiry date of the specific device, and date of manufacture. The repository reflects a possible compromise of all deployed appliances based on the model, typically by an optical scanner for bar coding of the identifiers. A centralized database and server compares the received identifier with the repository and reports, if a match was found between the received identifier and the anomalies in the database, of a possible compromise of the appliance.

Medical device quality issues impact every stakeholder in the medical device value continuum, including the manufacturer, the healthcare providers and user facilities, payers and, most importantly, the patients. Arguably, one of the most important facets of a manufacturer's Quality Management System (QMS) is their Post Market Surveillance (PMS) which contains processes and activities to monitor commercialized medical device performance. Given that the medical device industry is one of the most closely regulated industries, there are FDA and ISO regulations in place which provide a strict set of expectations on how manufacturers must operate in this regard. There are two main categories of PMS: (1) reactive methods which include complaint handling and (2) proactive methods which include surveys and Post-Market Clinical Follow-up. Even with regulations in place, medical device performance data collection (e.g., complaints) has been a pain point across the industry due to major inefficiencies and gaps within the process, resulting in large financial and human costs. It has been shown that remediation activities and external quality failures are major drivers for the direct cost of poor quality. Remediation activities include investigations, corrective and preventative actions (CAPAs), complaints, and field actions. Substantial financial impact is associated with tracking and remediating medical device anomalies, particularly those causing harm, and redress of harm-causing anomalies can be substantial.

In particular configurations, a system, method and dedicated apparatus or computing device provide a method for determining safe medical devices by deploying and receiving an identifier of a medical device under consideration for deployment, meaning a packaged device at a point of care for eminent use with a patient. The system compares the identifier, typically a bar or square coded string of alphanumeric digits, to a set of entries in a repository, and determining, based on at least one of a current usage attempt and the comparison, anomalies precluding safe device operation.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a context diagram of a patient treatment environment suitable for medical device use as disclosed herein;

FIG. 2 is a control flow of device introduction in the environment of FIG. 1;

FIG. 3 is a control flow for reporting a usage result of the device of FIG. 2;

FIG. 4 is a control flow of future activity with respect to the device of FIG. 2;

FIG. 5 is a GUI screen for entry of a user/reporter of medical device performance in the environment of FIG. 1;

FIG. 6 is a GUI screen for entry of a patient recipient of the medical device(s) of FIG. 2;

FIG. 7 is an option screen for medical device identification in the environment of FIG. 2;

FIG. 8 is a GUI screen for confirmation of medical device identity of the medical device of FIG. 7;

FIG. 9 is a warning screen for detected anomalies of the medical device of FIG. 8;

FIG. 10 is a GUI rendering of a report submission pertaining to the medical device of FIGS. 2-8;

FIG. 11 is a reporting screen for analytics of device performance across multiple medical facilities;

FIG. 12 is a reporting screen for analytics depicting device performance at an individual medical facility; and

FIG. 13 is an entry screen for Post Market Clinical Follow-up (PMCF) facilitating proactive collection of customer feedback relating to medical device performance.

### DETAILED DESCRIPTION

The configurations described below provide more efficient collecting, analyzing, and communicating of performance information of medical devices. More specifically, the technology is a dynamic platform that focuses on collecting complete and error-free user-inputted data and automatically outputting important medical device analytics for the user, which is intended to improve patient safety and clinical outcomes. Additionally, the user receives data-driven alerts in a timelier fashion than current methods which will allow them to take appropriate action, reducing patient risk opportunities. Furthermore, the system will allow medical device traceability and trackability.

Medical device complaints about adverse and harmful effects are typically reported in a static, ad-hoc method, in which many complaints are submitted with incomplete or incorrect information through either one of the following methods: calling the complaint to the manufacturer's sales representative, filling out the hardcopy or electronic FDA MedWatch Form, filling out an electronic complaint form directly on the manufacturer's website, or calling the complaint directly to the manufacturer's complaint call center. Consequently, the complaint data is typically ineffective in capturing important risk information (e.g., the exact severity of the harm) relating to the International Standards organization (ISO) 14971:2019 standard that medical device manufacturers are expected to comply with.

Conventional medical device manufacturers are unable to address complaint allegations in a real-time manner, and when they finally do, they often need to contact the alleger for additional information. Unfortunately, timely gathering of relevant information often does not occur. This results in an incomplete or inaccurate complaint data set for the manufacturer to take appropriate action on, preventing the ability to properly trend device performance analytics, find, and resolve a potential problem.

Specific issues of the complaint data submission with conventional methods include but are not limited to the following: device event descriptions are too vague (e.g., allegation only states that the device malfunctioned), the current complaint forms focus attention on just the device that failed when multiple devices are typically used interoperably and interfacing with each other, resulting in undocumented information, and that the device identification information is missing/incorrect. The cost to process a complaint for medical manufacturers and the reporters is substantial due to the amount of resources and time needed to complete an investigation. At present, US medical facilities are only required to report device-related deaths or serious injuries; therefore, many complaints do not get formally documented and reported, and may never reach the medical device manufacturer.

A further impediment is that health care providers (HCPs), medical device manufacturers, and the general public lack access to holistic and efficient medical device performance data. The FDA provides a database purported to assimilate device anomalies (Manufacturer and User Facility Device Experience, or MAUDE) for redress of this problem. However, the FDA MAUDE database captures only the Medical Device Reports (MDRs) that get published in a raw, text format which makes it difficult and time-consuming to analyze the data. Furthermore, this database is only updated monthly, so this critical information may not be timely available. An MDR may relate to any adverse event or anomaly that occurs during the use of the device or has the potential to recur. Therefore, in practice only a fraction of the complaints ever reaches this FDA database. It would be beneficial to provide a more efficient, real-time, and holistic database.

When a medical device is recalled, patients and users may not be informed in a timely manner to avoid recalled devices being used on successive patients. The current methods for medical device manufacturers to notify their customers of a recalled device is highly inefficient, involving an ad-hoc process of many resources and extensive time. Once a manufacturer identifies a device that may pose a health risk to the public, they may voluntarily send a safety alert to their customers and take steps to reach others who need to be notified. It then typically takes about another month for the FDA to assess the information provided by the manufacturer and designate a recall classification.

If appropriate, the FDA will oversee the recall and ensure that the manufacturer is taking appropriate action. Additionally, if a device manufacturer fails to recall a device voluntarily, the FDA has the authority to impose a mandatory recall. Even if the user facilities are alerted immediately on the recall, there is opportunity for human error since a hospital employee still needs to go into the storage areas and manually identify the devices that are in scope of the recall by reading the label of each device and then removing them from the facility. There is a need for a more efficient medical device recall notification system that notifies the user at the point of care to prevent recalled devices from being used as well as quickly identify and alert the user about previous device cases where a now recalled device had been used.

Europe has similar parallels. The European Union Medical Device Regulation (EU MDR) is requiring any medical device manufacturer that is marketing and selling devices in any of the European Union countries to have a proactive process and plan in place to collect medical device performance information and enhanced device traceability through Unique Device Identifiers (UDI). The result is increased costs for the medical device manufacturer, amounting to up to 5% of their revenue to address the new requirements. As a result, some companies are forced to stop distribution of their devices in these countries or altogether refrain from selling devices in these countries.

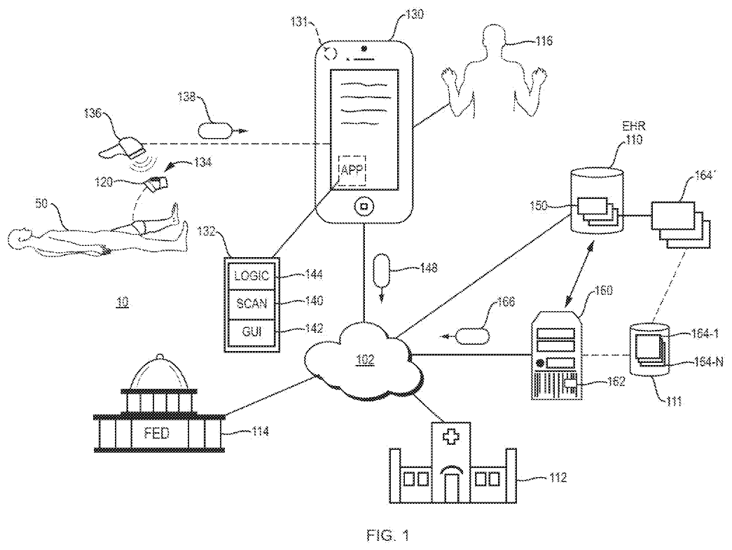

FIG. 1 is a context diagram of a patient treatment environment suitable for medical device use in the system and method as disclosed herein. In a medical treatment environment 10, an illustrative example includes a patient 50 recipient of a medical device 120. Any suitable medical device could be provided in the present approach; the illustrative example is a prosthetic knee. The patient 50 has a patient record 150 in an EHR (Electronic Healthcare Records) database and system 110, which employs a public access network 102 and aggregates the patient's medical history including information from healthcare facilities 112 and provides adherence to health records reporting and privacy standards, typically promulgated by a governmental entity 114 such as the FDA and HIPPA (Health Insurance Portability and Accountability Act). In the proposed approach, a reporter 116, such as a doctor, nurse, or healthcare provider operates a portable device 130 having an app 132 implementing a device reporting tool (DRT). Any suitable computing device may host the app 132, such as a personal smartphone device, tablet, laptop or PC.

The reporter locates the medical device 120 for use or implantation. The method disclosed herein for determining safe medical devices includes receiving an identifier of a medical device under consideration for deployment. Each medical device has a unique identifier 134, typically in the form of a labeled barcode, along with a vendor or manufacturer. Other designations, such as a model, device type, and serial No. may also appear, although these may all be determined from the unique identifier 134 if not immediately present on the device 120. The identifier 134 may be scanned 138 by a scanner device 136, retrieved from a photographic image taken via a camera 131 feature of the reporter's device 130, or simply entered in alphanumeric characters.

The DRT app 132 includes instructions for scanning 140, GUI presentation 142, and comparison/reporting logic 144. Complementing the app is a server 160, such as a cloud server or dedicated CPU, executing tracking logic 162 in conjunction with the device app 132. The tracking logic 162 compares the identifier 134 to a set of entries in a repository 110, and determines, based on at least one of the current usage attempt and the comparison, anomalies precluding safe device operation. The device entries may be part of the EHR and repository 110 or may be a separate repository, discussed further below. A message 166 indicates whether matching anomalies were found. If a similar device (same type or model) has experienced an anomaly, the personal device 130 will render an indication of the same. Similarly, if no derogatory anomalies are found, and device use/implantation continues, adverse events are recorded in the repository 110 to alert other subsequent users, thus marking the “first” anomaly. The repository 110 is preferably integrated with the EHR, however may also be maintained in an independent repository.

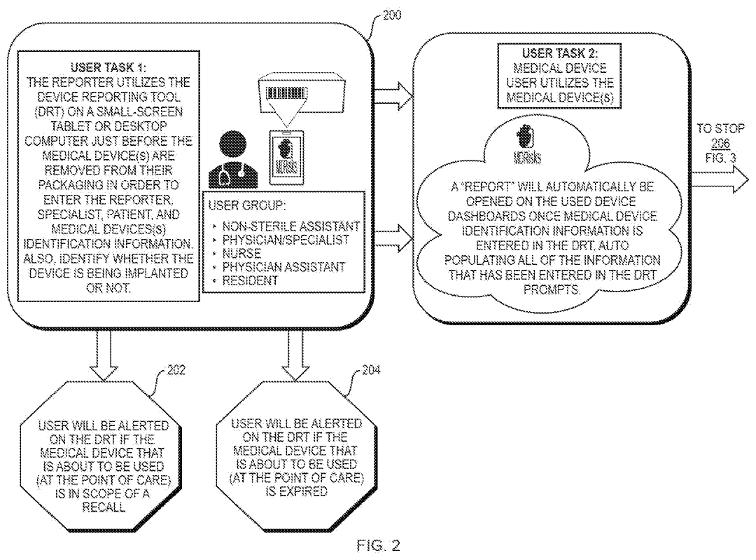

FIG. 2 is a control flow of device introduction in the environment of FIG. 1 Referring to FIGS. 1 and 2, in a medical device usage as in FIG. 1, the operator initiates reconciliation of safe medical device operation based on current and previous anomaly free operation, as depicted at step 200. Reconciliation refers to both prior usage anomalies and anomalies with the present usage. Using the device identifier 134, the app 132 first ensures that the set of entries in the repository 110 is free from reported device anomalies corresponding to the medical device. If prior anomalies are relevant, a GUI message informs the operator 116 for a recall, as shown at step 202, or of an expired device, at step 204. Passage of an expiration date of a sold or distributed medical device is considered an anomaly. Otherwise, if no anomalies are found, based on entries in the repository 110, then the personal device 130 receives an indication 166 of safe operation of the medical device during the current usage attempt.

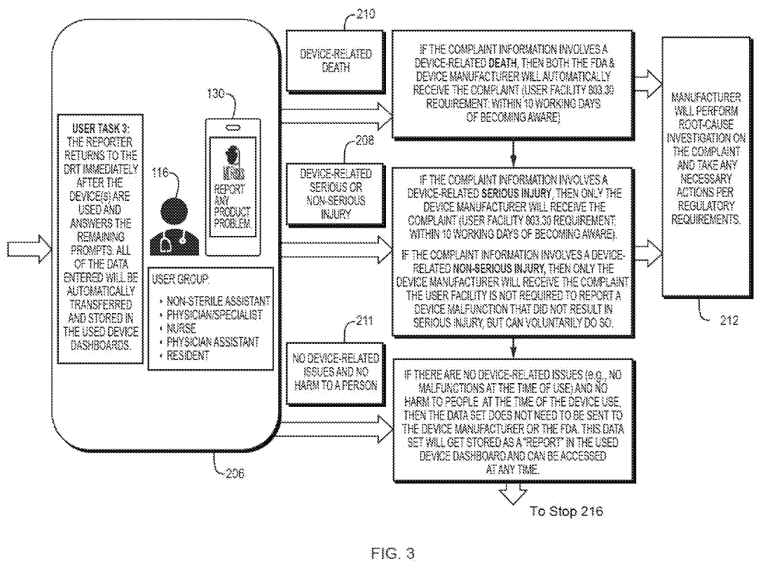

FIG. 3 is a control flow for reporting a usage result of the device of FIG. 2. Referring to FIGS. 1-3, if no previously reported anomalies preclude device usage, as in steps 202 or 204, usage and/or implantation proceeds. Upon completion, the app 132 receives an indication of an anomaly encountered during the current usage attempt, if any, as shown at step 206. The reportable anomalies are indicative of either minor harm or a serious injury, at step 208, or a fatality (step 210) resulting from the usage attempt. Successful, noneventful usage involves no needed reporting, at step 211. Otherwise, the app 132 generates an entry 164 in the anomaly repository 111 indicative of the encountered anomaly. Alternatively, the anomaly repository 111 may extend from the EHR repository 110 and associate the device anomaly entries 164 with the corresponding patient record 150 as an associated entry 164′. This allows the device anomalies to be stored and processed with the patient record of the patient experiencing the anomaly or failure. The generated entry 164 is then available for subsequent comparisons, depicted at step 212.

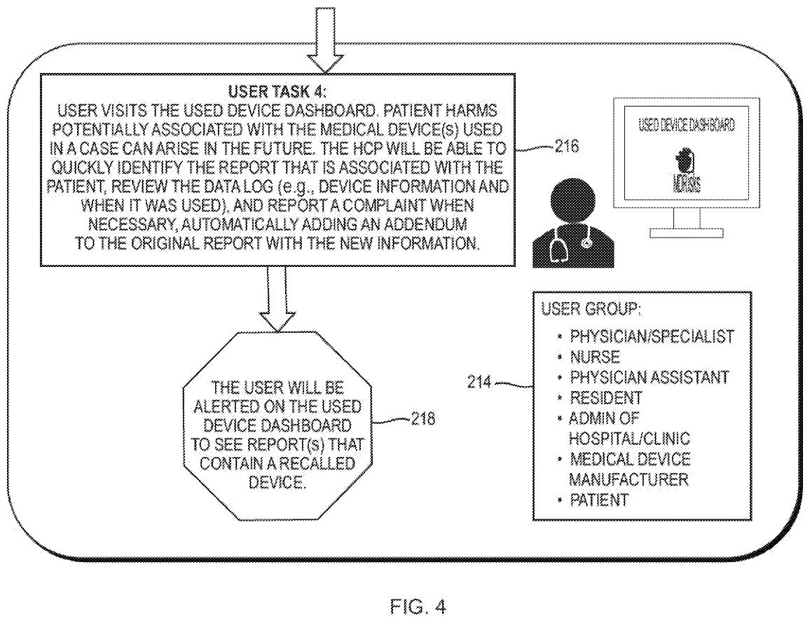

FIG. 4 is a control flow of future activity with respect to the device of FIG. 2. Referring to FIGS. 1-4, a device dashboard rendering is accessible to users 214 based on previously used devices, as shown at step 216. Access is provided based on appropriate privacy, such as a patient viewing their devices used on the patient's behalf or implanted into the patient, or doctors that used/implanted the devices. Outstanding anomalies will be rendered upon a user login, as shown at step 218.

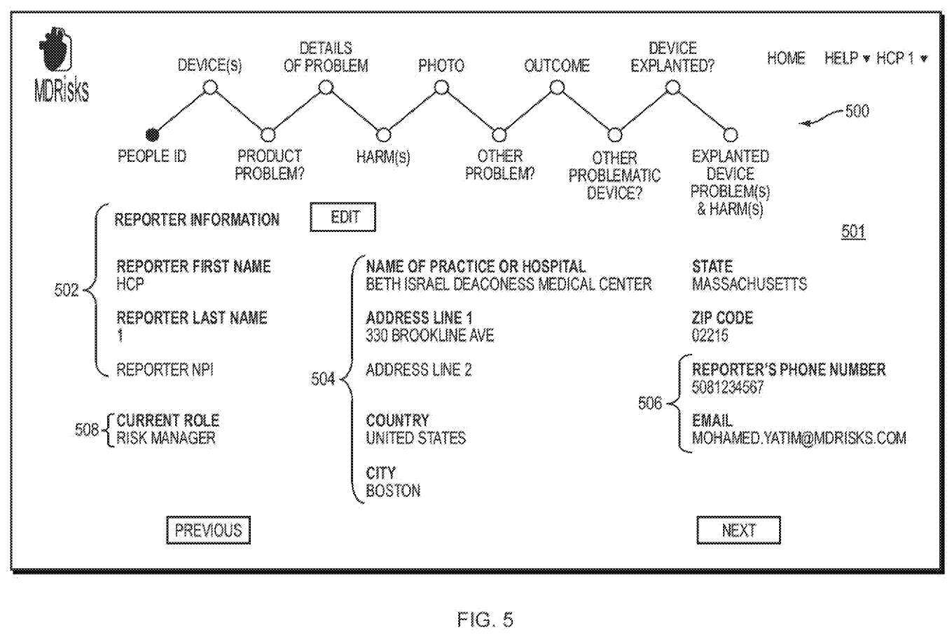

FIG. 5 is a GUI screen for entry of a reporter/operator 116 of medical device performance in the environment of FIG. 1. Referring to FIGS. 1-5, the GUI 142 drives a user experience as shown in FIGS. 5-12, as a device usage scenario is undertaken. FIG. 5 shows a reporter entry screen 501 for commencing a device usage. Recall that the reporter 116 is generally a healthcare provider administering the medical device, in contrast to the patient 50 upon whom the device is used. This is the party responsible for entry of the medical device 120 information, typically the circulating nurse or designated assistant, (e.g., non-sterile assistant, physician assistant, biomedical technician, or medical device manufacturer sales representative) depending on the nature of the medical device and operating room environment. The reported anomalies may range from a cause of patient death to a clerical error in labeling the device, so some usage contexts may not require direct oversight by a medical doctor. A further entry screen for gathering information specific to the specialist/doctor responsible for or administering usage/implantation is also entered, along with relevant information.

In traversing the entry sequence, a progress line 500 along the top of the screen margin shows darkened icons or circles as progress advances from screen to screen. Reporter information is collected at element 502, and medical facility information at 504 to designate the medical facility of usage. Contact information for the reporter is received at 506, and the current role or title at 508.

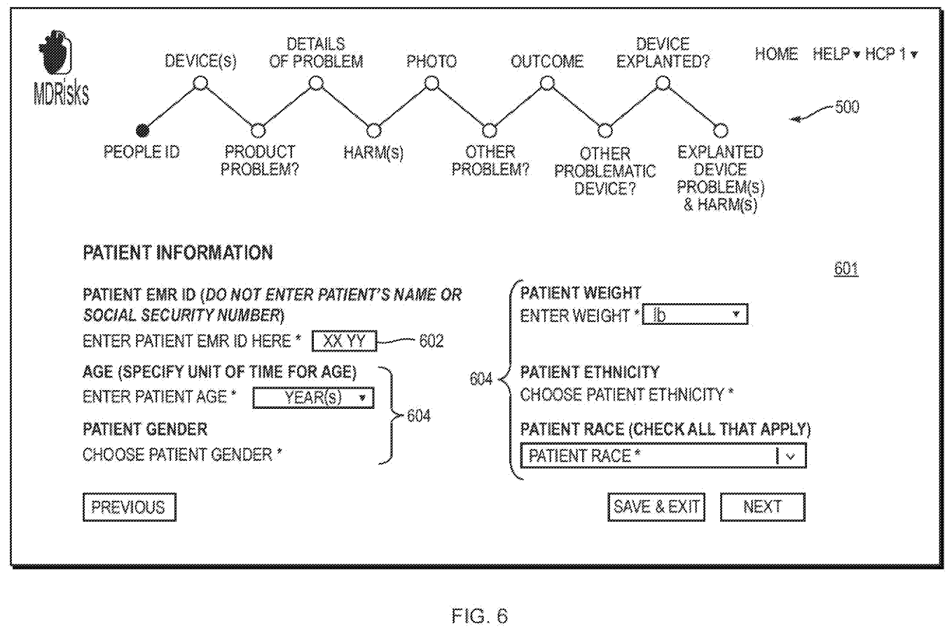

FIG. 6 is a GUI screen for the Device Reporting Tool (DRT) entry of a patient recipient of the usage/implant/operation of the device of FIG. 2. Referring to FIGS. 1-6, a patient entry screen 601 collects information on the patient, to associate the patient with the device usage and also with the preexisting patient record 150 in the EHR database 110. A patient Electronic Medical Record ID (EMR ID) is entered at 602 for identifying an entry 150 in the EHR (Electronic Health Records) system for the patient 50. The EMR ID is used to identify the patient record 150 in the EHR corresponding to a usage of the medical device. Other patient information 604 is also requested, and the generated entry in the patient record 150 in the EHR 110, soon to be followed by the medical device to be used. Multiple devices may be entered for a particular procedure or transaction. Typically, an implanted device is also accompanied by surgical and/or implanting devices that are used in the procedure to implant the device that remains following the procedure. Entry of multiple devices used in conjunction with each other helps to identify interoperability problems between devices, discussed further below.

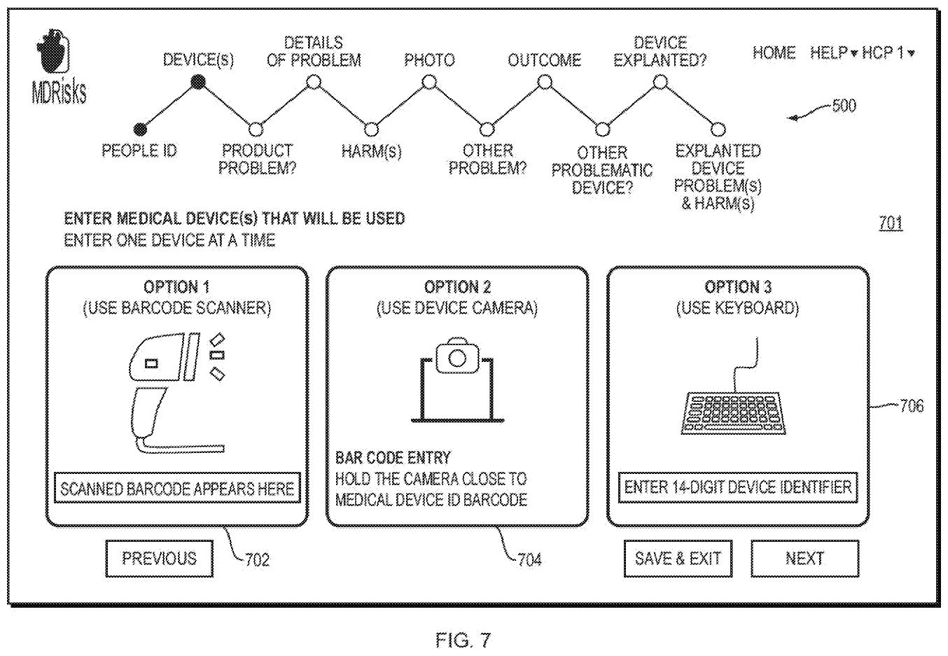

FIG. 7 is an option screen for medical device identification in the environment of FIG. 2. Referring to FIGS. 1 and 7, each medical device has an identifier 134 in some form, typically a label or printed bar code and corresponding alphanumeric string. The unique identifier 134 is an industry recognized unique identifier for the medical device and can be used to derive the vendor and model, if such information is not also prominently displayed. The app 132 receives the identifier of the medical device from one of an optical reader 136 scanning a bar code on the medical device, a photographic image of the medical device from the personal device 130 on which the app 132 resides, or alternatively, the 14-digit device identifier 134 can be manually entered from an alphanumeric rendering on the medical device.

The identifier screen 701 includes option icons for the selected input mechanism, including a scanner icon 702 to invoke the scanner 136 and scan logic 140 of the app, a device camera icon 704 to recognize the bar code or square code using a photographic image from the camera 131 of the device, or a keyboard icon 706 for manual entry using a keyboard capability of the device 130. Whatever the form of entry, a message 138 including the identifier is generated for comparison.

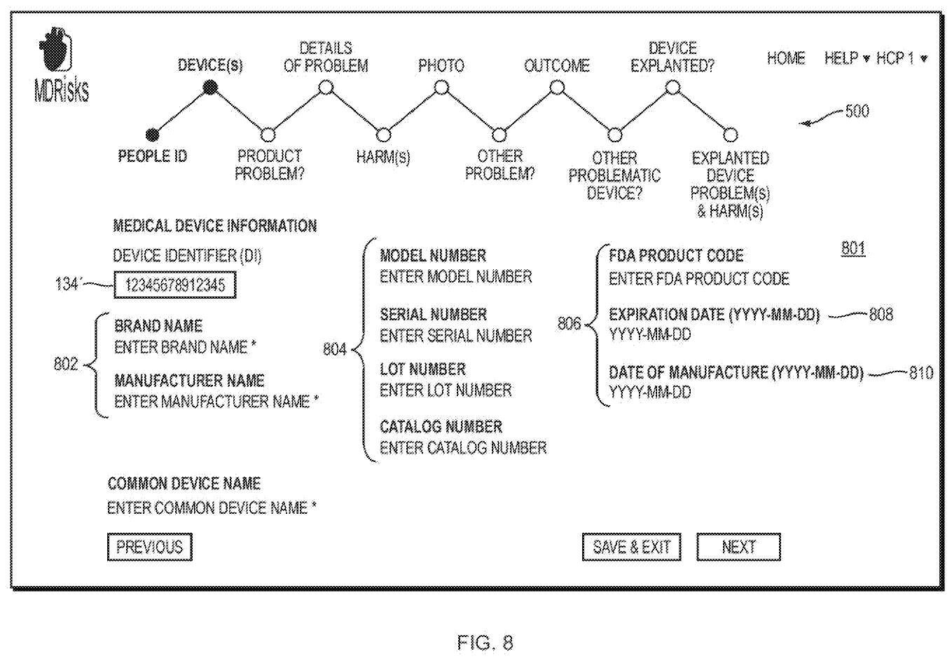

FIG. 8 is a GUI screen 801 for confirmation of medical device identity of the medical device of FIG. 7. The alphanumeric rendering of the device identifier 134′ is shown. Manufacturer information can either be manually entered or retrieved via the device identifier 134. Similarly, the device information fields 804 may be manually entered or retrieved based on the transmitted identifier 138. Regulatory/governmental based fields 806, such as the FDA for US based devices, is also rendered. The expiration date 808, which is based on the expected longevity of the device, and date of manufacture 810.

The server 160 generates the entry 164-1 . . . 164-N (164 generally) in the repository 110, 111 including the related fields for the medical device 120, and will append a subsequent response to an anomaly encountered in the current usage attempt. An anomaly of a device may indicate a potential for harm to other patients using or receiving another similar medial device. In the event an anomaly results in a recall or other widespread concern, a subsequent usage involves receiving an identifier and a request for a second comparison of a second medical device, where the second medical device is the same or similar type as the first medical device experiencing the anomaly. A similar type would have the same model number or name, and may be limited to a particular batch, lot number or manufacturing run. Alternatively, similar models having a different model number but sharing similar features or parts might also be triggered by an anomaly. The reporting of an anomaly is intended to commence action in determining a cause and result of the anomaly, and depending on the scope of the determined cause, other affected medical device units can be determined. By having deployed medical devices stored in a respective entry 164, the capability to identify patients of previous procedures involving the affected device, and immediately flag subsequent usage attempts, avoids the delays and overhead of conventional approaches to mitigating harmful results. The app 132, based on the comparison logic 144, can immediately render an indication for preventing usage of the second medical device based on the second comparison, meaning the eminent usage following an identified anomaly.

Governmental entities, such as the FDA, may also mandate reporting of certain anomalies. The reporting logic is further configured to identify an interface to a report gathering authority including at least one of a manufacturer and a governmental entity. Reports to a government entity would also be duplicated to the manufacturer and likely the vendor, if they differ. Depending on the level of harm, the reporting logic determines if the device related event is a serious adverse event; and if so, transmits the device related event to the appropriate party via the identified interface.

The repository 110 arranges the set of entries 164 according to a vendor and a model, in which the model designates a type of the medical device. Accordingly, upon scanning/identifying a medical device 120 for which usage is eminent, the server 160 identifies, based on the received identifier 138, a model of the medical device, and traverses entries in the set of entries corresponding to the model for determining anomalies encountered with the same model of the medical device.

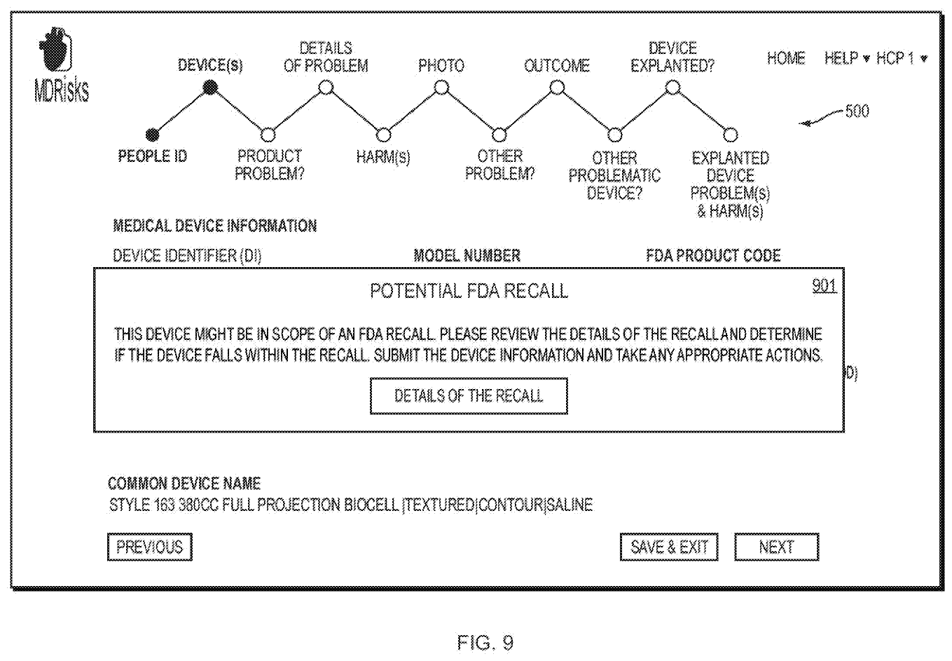

FIG. 9 is a warning screen for detected anomalies of the medical device of FIG. 8. After comparing the device identifier 134 with the recalled devices, an alert screen 901 renders, based on the comparison, whether the device has been the subject of a reported anomaly indicative of unsafe usage. A similar screen indicates an expired device, based on a comparison of the expiration date and manufacturing date.

If the device is not the subject of known problems or recalls, attempted usage proceeds. If an anomaly occurs, a series of screens and prompts assists in generating a report 148 of the anomaly. If normal, uneventful usage occurred, no anomalies need be reported, and/or a report indicating an expected outcome occurs, including general parameters such as whether a device was explanted. Otherwise, entering the details of the anomaly occurs through a series of screens and entered fields, denoted by the progress line 500. In the example configuration, the operator 116 returns to the GUI 142 and proceeds according to screen guidance as follows

1) Return to the DRT and complete either of the following:

a) Indicate that there is No Product Problem, if a device was explanted, and record if there are any harms associated with the explanted device or non-medical device related event, and then click submit. The Used Device Report is now complete; or

b) Select a device to Report Any Product Problem (e.g., Defects or Malfunctions) Related to the Medical Device, Packaging, or Labeling. If a device fails in the middle of the case/usage and a replacement device is introduced, then the new device is added into in the DRT by entering the device information prior to opening its package.

Next, the operator enters a category and specific details of the medical device anomaly.

2. Once a device is selected for reporting of an anomaly, the operator 116, select the general category of the problem:

a) Labeling (Instructions for Use or on Packaging)

b) Packaging

c) Medical Device

The DRT is designed to allow the reporting of an additional problem for the same device by iterating as above until all problems for the device have been captured.

3. If the problem is labeling or packaging related, the operator 116 identifies the component that had a problem, the problem, and describe the event/product problem in detail, as the device itself may not lack integrity.

4. If the problem is medical device related, then describe the medical device problem by indicating what component(s) of the medical device had a problem and what problem was experienced.

The description of the device anomaly in steps 3 and 4, is a narrative description intended to focus on the device and not the harm/consequence. It should report any event(s) associated with the product, whether it results in harm or not. It should also report even if uncertainty exists that the product caused the event, or even if details are incomplete—notice is a primary objective. Typical entries pertain to quality, performance or safety concerns such as:

a) Suspected counterfeit product

b) Suspected contamination

c) Questionable stability

d) Defective/failed component(s)

e) Therapeutic failure(s) (product didn't work)

NOTE: Indicate the potential root cause when describing the event/product problem (e.g., use error, design failure). An event/product problem may result in people, environmental, property, or no harm, but these aspects of harm are reported below.

5. Identify the person(s) that experienced a harm caused by the product problem. A button list for no harm, patient harm, and/or user harm is presented.

6. Answer the questions relating to the harm(s) to the people associated with the product problem, using a button list of non-serious, serious, and death.

7. Describe the harm(s) (people, property, and/or environment) associated with the product problem in as much detail as possible in a narrative entry.

8. The GUI 142 accommodates an upload a photo of the product problem if relevant and available, using either an onboard camera or drag/drop file interface, depending on the device 130.

9. Identify the outcome of the problem(s) associated with the device. A bullet list summarizing the overall result includes options for:

a) Device utilized/implanted and procedure completed

b) Device exchanged

c) Procedure aborted and rescheduled

d) Required intervention to prevent permanent impairment/damage

e) Other

10. Is the Device Available for Evaluation? A bullet screen for affirmative availability is presented.

Iterations occur for reporting additional problem with the subject device.

11. Indicate whether or not there are any additional medical devices with problem(s). If so, iterations occur for additional medical devices. Following a usage resulting in an anomaly, other devices may also be relevant. Many medical devices are used in conjunction with other medical devices, and interoperability issues could be the cause of the anomaly.

12. Was a Device Explanted? This indicates whether the overall procedure using all concerned medical devices resulted in a device being explanted. Recall that some anomalies may merely be labeling and other non-invasive based problems.

The above description and report entry steps are illustrative. The range of possible reporting scenarios ranges from clerical inaccuracies to patient death, so some reports necessarily require more depth. From an industry perspective, a widespread usage of reporting of all devices provides a robust base from which to mitigate any harmful effects, but also to proactively confirm a likely absence of the same.

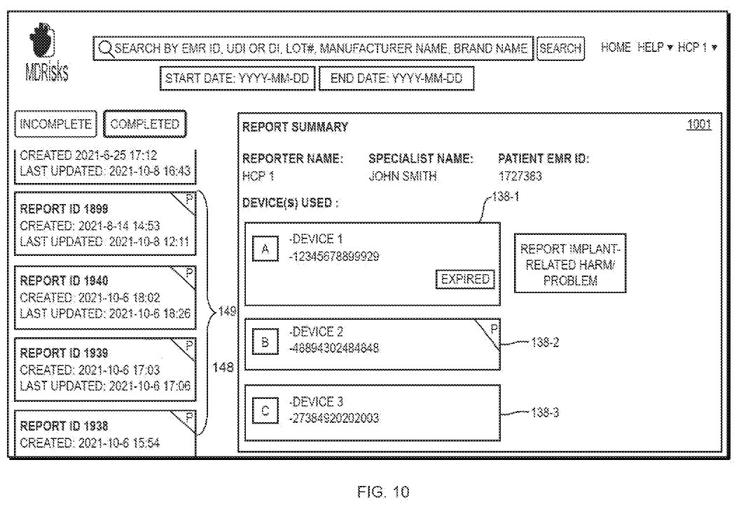

FIG. 10 is a GUI rendering of a report submission pertaining to the medical device of FIGS. 2-9. Upon completion of report entry discussed above, a report screen 1001 confirms the report 148 of one or more medical devices 120 involved with that case. The report screen 1001 accumulates a chronological set of entries 149 based on receiving a plurality of usage reports 148, such that each usage report 148 of the plurality of usage reports is based on a usage attempt and including the identifier 138-1 . . . 138-N (138 generally) indicative of the medical device and whether the usage attempt encountered normal operation or an anomaly. The server 160 accumulates the reports 138 in an entry 164 in an anomaly repository 111; alternatively, this may be an extension of the patient record 150 through the EHR 110 repository, such that the EHR also maintains the entries 164 based on the patients upon which the device was used or implanted. This coalesces the device reports 148 with the patient record 150 by associating the entries 164 with the corresponding patient record 150 maintained by the EHR 110.

The reporting logic is further configured to reconcile subsequent anomaly free operation accessing the set of entries in the repository for identifying patients associated with the medical device and transmitting a proactive inquiry for identifying anomalies presented subsequent to a usage of the medial device. In the event that post-procedure follow-up is either mandated or voluntarily undertaken, such data is gathered as a report. Manufacturers/vendors utilize the usage trail provided by the device and patient records to track the medical device after it has been utilized.

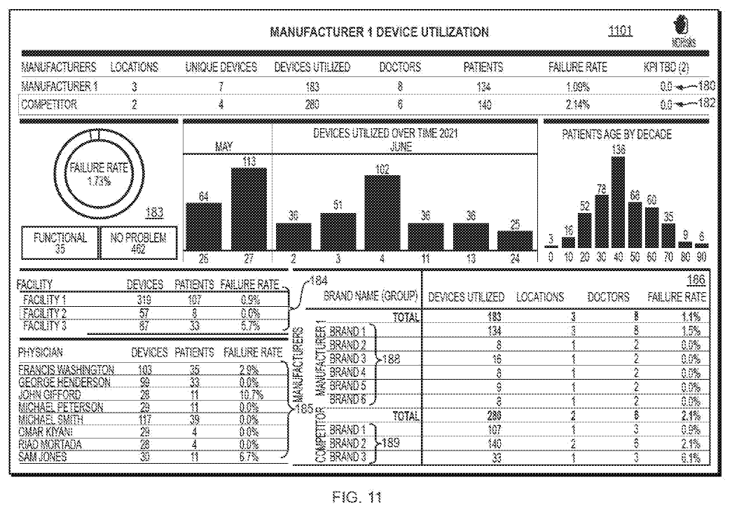

FIG. 11 is a reporting screen for analytics of device performance across multiple medical facilities. The collective entries 164 of device reports provides analytics for pinpointing facilities, manufactures, or practitioners associated with an outlier number of anomalies. An example analytics report 1101 illustrates failures from anomalies in the scope of a device utilization. By implementing a robust accrual of usage reports 148, for both normal usage and those with anomalies, a true device utilization emerges that accounts for devices sold but not incorporated in a patient usage context. Conventional failure analysis that tracks identified failures based on units sold could miss an emerging problem when a disproportionate failure rate emerges among a relatively small number of usages while numerous units remain warehoused at distributors or hospital stock.

In FIG. 11, an analytics request is received to render one or more of the entries 164 in the set of entries 164-N. A vendor entry 180 identifies a vendor corresponding to a source of the request, and competitor vendors are collectively grouped in a competitor entry 182 so as not to allow one vendor access to proprietary information of another. The report screen 1101 therefore renders only the entries from the set of entries corresponding only to the identified vendor initiating the request.

From the report screen 1101, an overall failure rate 183 is shown graphically. A facilities window 184 shows facilities where the reported devices were used, and a practitioner window 185 shows responsible doctors. Since a vendor/manufacturer may contractually distribute under different brand names, a brand names window 186 is also broken out by the initiating vendor 188 and competitor brand names 189, again to avoid dissemination of proprietary information. Such “genericizing” guards against manufacturers interrogating their competition and instead report statistical data only about how the requesting manufacture is faring. This improves safety because manufacturers will not tend to interpret or minimize anomalies to avoid reporting for fear of predatory competitors leveraging safety data for profit motives.

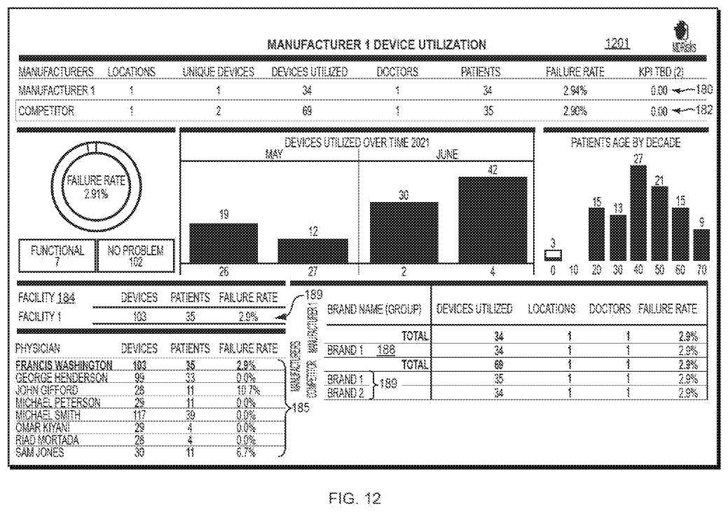

FIG. 12 is a reporting screen for analytics depicting device performance at an individual medical facility. In a facility specific screen 1201, device anomalies occurring at a specific medical facility are shown. The facility window 184 filters on a single facility, and the practitioner 185 and brand names window 186 reflect only the queried facility. This allows drilling down to identify facilities associated with a disproportionate rate of anomalies. Similar filtering can be performed for practitioners, brand names and the like, however not to single out a particular competitor.

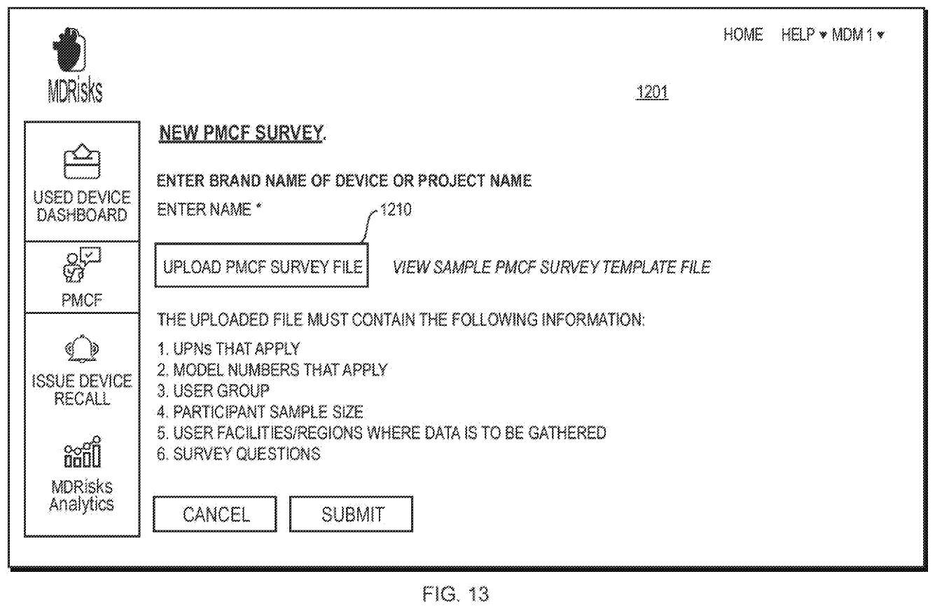

FIG. 13 is an entry screen for Post Market Clinical Follow-up (PMCF) facilitating proactive collection of customer feedback relating to medical device performance. Conventional approaches provide an inefficient capability or duty for device vendors to seek and confirm positive outcomes, short of an outright failure. Accordingly, a PMCF icon is selectable to access a PMCF electronic tool. When the PMCF icon is selected on the home screen of the Medical Device Manufacturer Account, the authorized user type can either create a new PMCF survey, view the summary table, or access the surveys and results that have already been created. Upon selection, the app renders a PCMF screen 1201, where the vendor uploads survey data and other relevant information via an upload button 1210 attesting to post-device usage success and/or patient wellbeing.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for determining safe medical devices, comprising:
receiving an identifier of a medical device, the medical device under consideration for deployment;
comparing the identifier to a set of entries in a repository; and
determining, based on at least one of a current usage attempt and the comparison, anomalies precluding safe device operation.

2. The method of claim 1 further comprising reconciling safe medical device operation based on current and previous anomaly free operation by:
ensuring the set of entries in the repository is free from reported device anomalies corresponding to the medical device; and
receiving an indication of safe operation of the medical device during the current usage attempt.

3. The method of claim 1 further comprising:
rendering, based on the comparison, whether the device has been the subject of a reported anomaly indicative of unsafe usage.

4. The method of claim 1 further comprising:
receiving an indication of an anomaly encountered during the current usage attempt; and
generating an entry in the repository indicative of the encountered anomaly, the generated entry available for subsequent comparisons.

5. The method of claim 4 further comprising:
generating the entry in the repository based on a first medical device in response to an anomaly encountered in the current usage attempt;
receiving an identifier and a request for a second comparison of a second medical device, the second medical device being the same type as the first medical device; and
rendering an indication for preventing usage of the second medical device based on the second comparison.

6. The method of claim 1 wherein the anomalies are indicative of either minor harm, serious injury or a fatality resulting from a previous usage attempt.

7. The method of claim 1 further comprising:
accumulating the set of entries based on receiving a plurality of usage reports, each usage report of the plurality of usage reports based on a usage attempt and including an identifier indicative of the medical device and whether the usage attempt encountered normal operation or an anomaly.

8. The method of claim 7 further comprising arranging the set of entries according to a vendor and a model, the model designating a type of the medical device, further comprising:
identifying, based on a received identifier, a model of the medical device; and
traversing entries in the set of entries corresponding to the model for determining anomalies encountered with the same model of the medical device.

9. The method of claim 1 wherein the identifier is an industry recognized unique identifier for the medical device.

10. The method of claim 4 further comprising:
identifying an EHR (Electronic Health Records) system;
identifying a patient record in the EHR corresponding to a usage of the medical device;
storing the generated entry in the patient record in the EHR.

11. The method of claim 1 further comprising:
receiving a request to render one or more of the entries in the set of entries;
identifying a vendor corresponding to a source of the request;
rendering the entries from the set of entries corresponding only to the identified vendor.

12. The method of claim 1 further comprising receiving the identifier of the medical device from one of an optical reader scanning a bar code on the medical device, a photographic image of the medical device from a personal device, or a typed alphanumeric identifier from a label on the medical device.

13. A system for gathering and tracking medical device reports for determining safe usage of medical devices, comprising:
a mobile device application for receiving an identifier of a medical device, the medical device under consideration for deployment;
a server for comparing the identifier to a set of entries in a repository; and
reporting logic for determining, based on at least one of a current usage attempt and the comparison, anomalies precluding safe device operation.

14. The system of claim 13 wherein the reporting logic is configured to reconcile safe medical device operation based on current and previous anomaly free operation by:
ensuring the set of entries in the repository is free from reported device anomalies corresponding to the medical device; and
receiving an indication of safe operation of the medical device during the current usage attempt.

15. The system of claim 13 further comprising GUI logic configured to render, based on the comparison, whether the device has been the subject of a reported anomaly indicative of unsafe usage.

16. The system of claim 13 wherein the server is further configured to:
receive an indication of an anomaly encountered during the current usage attempt; and
generate an entry in the repository indicative of the encountered anomaly, the generated entry available for subsequent comparisons.

17. The system of claim 16 wherein the server is further configured to:
generate the entry in the repository based on a first medical device in response to an anomaly encountered in the current usage attempt;
receive an identifier and a request for a second comparison of a second medical device, the second medical device being the same type as the first medical device; and
render an indication for preventing usage of the second medical device based on the second comparison.

18. The system of claim 14 wherein the reporting logic is further configured to reconcile subsequent anomaly free operation by:
accessing the set of entries in the repository for identifying patients associated with the medical device;
transmitting a proactive inquiry for identifying anomalies presented subsequent to a usage of the medial device and
receiving an indication of whether anomalies occurred post-usage or post-implantation.

19. The system of claim 13 wherein the reporting logic is configured to
identify an interface to a report gathering authority including at least one of a manufacturer and a governmental entity;
determine if the device related event is a serious adverse event; and if so,
transmit the device related event to the appropriate party via the identified interface.

20. A computer program embodying program code on a non-transitory medium that, when executed by a processor, performs steps for implementing a method for determining safe medical devices, the method comprising:
receiving an identifier of a medical device, the medical device under consideration for deployment;
comparing the identifier to a set of entries in a repository; and
determining, based on at least one of a current usage attempt and the comparison, anomalies precluding safe device operation.
